(19) United States
(12) Patent Application Publication
(10) Pub. No.: US 2026/0166528 A1
(43) Pub. Date: Jun. 18, 2026

(54) CATALYST MIXTURE AND METHOD OF SYNTHESIZING ALKANES AND OLEFINS USING THE CATALYST MIXTURE

(71) Applicant: INDUSTRIAL TECHNOLOGY RESEARCH INSTITUTE, Hsinchu (TW)

(72) Inventors: Kuo-Ching WU, Hsinchu City (TW); Pao-Tsern LIN, Zhubei City (TW); Hsi-Yen HSU, Hsinchu City (TW); Chao-Huang CHEN, Hsinchu City (TW); Cheng-Hsun LI, Guanxi Township (TW); Yen-Chih CHEN, Hsinchu City (TW); Ying-Chieh LEE, Hsinchu City (TW)

(73) Assignee: INDUSTRIAL TECHNOLOGY RESEARCH INSTITUTE, Hsinchu (TW)

(21) Appl. No.: 19/257,602

(22) Filed: Jul. 2, 2025

(30) Foreign Application Priority Data
Dec. 12, 2024 (TW) 113148366

(51) Int. Cl. B01J 23/889; B01J 23/745; C07C 2/86

(52) U.S. Cl. CPC B01J 23/8892; B01J 23/745; C07C 2/862

(57) ABSTRACT

Disclosed is a catalyst mixture that includes a mixture of a metal oxide catalyst and an iron-based catalyst. The metal oxide catalyst has a chemical formula of Fe₁MnₐM_{b}Oₓ, wherein M is Ce or La, a is 0.1 to 0.5, b is 0.1 to 0.3, and x is chemical stoichiometry. The iron-based catalyst includes 70 mol % to 97 mol % of porous FeO(OH)_{c}, wherein 1<c<2, and 3 mol % to 30 mol % of an alkaline metal compound loaded onto the porous FeO(OH)_{c}.

### CROSS REFERENCE TO RELATED APPLICATIONS

The present application is based on, and claims priority from, Taiwan Application Serial Number 113148366, filed on Dec. 12, 2024, the disclosure of which is hereby expressly incorporated by reference herein in its entirety.

### TECHNICAL FIELD

This application relates to a catalyst mixture and a method of synthesizing alkanes and olefins using the catalyst mixture.

### BACKGROUND

Processes of converting carbon dioxide and hydrogen into alkanes and olefins are known. Conventional processes consume a certain degree of hydrogen. However, hydrogen sources are limited, and an alternative source of hydrogen and a corresponding catalyst system are needed to further reduce the cost of converting carbon dioxide into alkanes and olefins.

### SUMMARY

One embodiment of the disclosure provides a catalyst mixture comprising a metal oxide catalyst and an iron-based catalyst. The metal oxide catalyst has a chemical formula of Fe₁MnₐM_{b}Oₓ, wherein M is Ce or La, a is 0.1 to 0.5, b is 0.1 to 0.3, and x is chemical stoichiometry. The iron-based catalyst includes 70 mol % to 97 mol % of porous FeO(OH)_{c}, wherein 1<c<2, and 3 mol % to 30 mol % of alkaline metal compound loaded onto the porous FeO(OH)_{c}. The “a” may be 0.1 to 0.43. The “b” may be 0.14 to 0.3. The “c” may satisfy 1<c≤1.52.

### DETAILED DESCRIPTION

Disclosed embodiments provide a novel catalyst mixture and process technology for synthesizing hydrocarbons, such as alkanes and olefins, from methane, hydrogen, and carbon dioxide. In particular, the inventors have found that according to embodiments, certain metal oxide catalysts and iron-containing catalysts may be combined into catalyst mixture and employed in selective reactions in which methane (CH₄) and carbon dioxide (CO₂) are recombined at unexpectedly low temperatures to provide alkanes (CₙH₂ₙ₊₂ having a carbon number of 2 or more) and/or olefins (CₘH₂ₘ having a carbon number of 2 or more) and water (H₂O). These catalyst mixtures and methods allow for surprisingly economical reduction in carbon footprint by, for example, efficiently converting CO₂ without consuming too much hydrogen relative to conventional methods.

A detailed description is given in the following discussion of disclosed embodiments.

In the following detailed description, for purposes of explanation, numerous specific details are set forth in order to provide a thorough understanding of the disclosed embodiments. It will be apparent, however, that one or more embodiments may be practiced without these specific details.

One embodiment of the disclosure provides a catalyst mixture including a mixture of a metal oxide catalyst and an iron-based catalyst.

The metal oxide catalyst has a general chemical formula of Fe₁MnₐM_{b}Oₓ, wherein M is Ce or La.

The “a” in the general formula may be in a range of 0.1 to 0.5, 0.11 to 0.49, 0.12 to 0.48, 0.13 to 0.46, 0.14 to 0.43, 0.2 to 0.4, 0.25 to 0.35, or 0.3 to 0.35.

The “b” in the general formula may be in a range of 0.1 to 0.3, 0.11 to 0.29, 0.12 to 0.29, 0.13 to 0.29, 0.14 to 0.28, 0.15 to 0.25, 0.18 to 0.23, or 0.19 to 0.21.

The “x” may be determined according to the resulting chemical stoichiometry, according to methods known in the art.

The iron-based catalyst includes 70 mol % to 97 mol % of porous FeO(OH)_{c}, an iron-based support, wherein 1<c<2, and 3 mol % to 30 mol % of alkaline metal compound loaded onto the porous FeO(OH)_{c}.

For the iron-based catalyst, if c=0, the iron-based support will be FeO (i.e., iron oxide), and the iron-based catalyst may not be capable of synthesizing the alkanes and olefins according to embodiments. In general, the iron-based support (e.g., the porous FeO(OH)_{c}) is substantially free of iron oxides such as FeO, Fe₂O₃, Fe₃O₄, or the like, which can be confirmed by the Raman spectrum of the iron-based support. In general, the iron oxide will deteriorate the performance of the iron-based catalyst (containing the iron oxide) in synthesizing alkanes and olefins. If the amount of alkaline metal compound is too low, the content of the intermediate product carbon monoxide may be too high. If the amount of alkaline metal compound is too high, the conversion rate of the carbon dioxide may be too low and the carbon dioxide may not be efficiently converted to alkanes and olefins.

In some embodiments, the metal oxide catalyst and the iron-based catalyst have a volume ratio of 1:4 to 1:1. If the volume of the metal oxide catalyst is too low, the methane may not react to form the alkanes and olefins. If the volume of the metal oxide catalyst is too high, the yield of the alkanes and olefins may be decreased.

In some embodiments, the porous FeO(OH)_{c} may have a specific surface area of at least 100 m²/g. The porous FeO(OH)_{c} may have a specific surface area in the range of 100 m²/g to 300 m²/g. If the specific surface area of the porous FeO(OH)_{c} is too small, the available reactive surface area may be too small, resulting in insufficient catalyst performance.

In some embodiments, the porous FeO(OH)_{c} has a pore volume of 0.2 cm³/g to 0.5 cm³/g. If the pore volume of the porous FeO(OH)_{c} is too small, the surface area and activity of the porous FeO(OH)_{c} may decline.

In some embodiments, the porous FeO(OH)_{c} has an average pore size of 40 Å to 70 Å. If the average pore size of porous FeO(OH)_{c} is too large, the surface area and activity of the porous FeO(OH)_{c} may decline.

In some embodiments, the alkaline metal compound may include sodium carbonate, potassium carbonate, sodium hydroxide, potassium hydroxide, sodium oxide, potassium oxide, or a combination thereof. In general, the alkaline metal compound is a common alkaline compound such as sodium carbonate, potassium carbonate, sodium hydroxide, potassium hydroxide, or a combination thereof. However, a part of the carbonate may decompose to carbon dioxide and oxides during the heating process, and a part of the hydroxide may dehydrate into oxides during the heating process.

In some embodiments, the iron-based catalyst may be formed by the following steps. First, an alkaline solution (such as ammonia water, sodium hydroxide aqueous solution, potassium hydroxide aqueous solution, sodium carbonate aqueous solution, or potassium carbonate aqueous solution) is rapidly mixed with an iron salt aqueous solution (such as iron nitrate or iron hydroxide aqueous solution). For example, the alkaline solution and the iron salt aqueous solution can be stirred at high speed (e.g., >1000 rpm) for a short period of time (e.g., less than 5 minutes) to be rapidly mixed. Alternatively, the rapid mixing can be performed by any other method and is not limited to stirring at high speed. If the mixing speed is not fast enough, the iron-based support will have a specific surface area that is too low, a pore volume that is too small, and an average pore size that is too large.

Subsequently, the pH value of the mixture is adjusted to 8 to 10 (e.g., 9) and rested for 1 to 3 hours in order to precipitate. If the pH value is too low, the particle size and the average pore size of the precipitate will be too large. If the pH value of the mixture is too high, the precipitate cannot be formed efficiently. The precipitate is then filtered out, and the filtered cake is washed with water and baked at 100° C. to 110° C. to obtain an iron-based support, i.e., FeO(OH)_{c}. If the alkaline solution is ammonia water, the iron-based support should be further immersed into an aqueous solution of an alkaline metal compound such as sodium hydroxide, potassium hydroxide, sodium carbonate, or potassium carbonate and then dried for loading the alkaline metal compound onto the iron-based support. If the alkaline solution is sodium hydroxide aqueous solution, potassium hydroxide aqueous solution, sodium carbonate aqueous solution, or potassium carbonate aqueous solution, the loading step can be optionally omitted. Finally, the iron-based support having the alkaline metal compound loaded thereon is dried and shaped to obtain the iron-based catalyst. If the baking and drying temperature is too high, Fe₂O₃ or Fe₃O₄ will be produced which deteriorate the performance of the iron-based catalyst. If the baking and drying temperature is too low, FeO(OH)_{c} will not be efficiently produced.

In general, the iron salt reacts with the alkaline to form iron hydroxide (Fe(OH)₃). After heating, an intramolecular dehydration reaction will occur for the iron hydroxide to form Fe₂O(OH)₄, which may further be dehydrated to form FeO(OH)_{c}. FeO(OH)_{c} can be further dehydrated to form Fe₂O₃ or Fe₃O₄. It will be understood from the TGA analysis spectrum that when the iron hydroxide (Fe(OH)₃) is heated to 50° C., the intramolecular dehydration occurs, which induces Fe₂O(OH)₄ formation. When Fe₂O(OH)₄ is further heated to about 100° C. to 110° C., FeO(OH)_{c} is formed. As shown in the Raman spectra, there are almost no signals of Fe₂O₃ and Fe₃O₄ in FeO(OH)_{c}. If FeO(OH)_{c} is further heated to a higher temperature (e.g., higher than 200° C.), Fe₂O₃ or Fe₃O₄ will be formed. The mole number of Fe and the number of c of the formed FeO(OH)_{c} can be calculated from the initial weight of the iron salt. For example, if the mole number of Fe is 1 mole and the whole product is FeO(OH), the product weight should be 88.85 g. If the mole number of Fe is 1 mole and the whole product is FeO(OH)₂, the product weight should be 105.86 g. As such, when the product in practice has a weight of 97.35 g, c number of the FeO(OH)_{c} will be about 1.5, i.e., FeO(OH) and FeO(OH)₂ have a molar ratio of about 1:1.

In some embodiments, the metal oxide catalyst can be formed by following steps. First, iron nitrate, manganese nitrate, and lanthanum nitrate (or cerium nitrate) were weighed according to chemical stoichiometry and dissolved in water, and a pH value of this aqueous solution of the metal salts was then adjusted by an alkaline liquid (e.g., ammonia water, sodium hydroxide aqueous solution, potassium hydroxide aqueous solution, sodium carbonate aqueous solution, or potassium carbonate aqueous solution) to 8 to 10 (e.g., 9) and kept for 1 hour to 3 hours to produce a precipitate. If the pH value of the aqueous solution was too low, the particle size of the precipitate will be too large. If the pH value of the aqueous solution is too high, the precipitate cannot be formed efficiently. Subsequently, the precipitate is collected by filtering, and the filtered cake is washed by water, baked, and then sintered at a high temperature (such as 400° C. to 500° C., e.g., 450° C.) to obtain a metal oxide catalyst Fe₁MnₐM_{b}Oₓ. It should be understood that the method of forming the metal oxide catalyst is merely an example. One skilled in the art may adopt any method to form the metal oxide catalyst and is not limited to the described method.

One embodiment of the disclosure provides a method of synthesizing saturated hydrocarbons such as alkanes and unsaturated hydrocarbons such as alkenes or olefins.

The catalyst mixture disclosed herein may be particularly suitable for synthesizing alkanes and olefins. According to embodiments, the synthesis reaction may proceed according to reaction Formula I or Formula II as shown below:

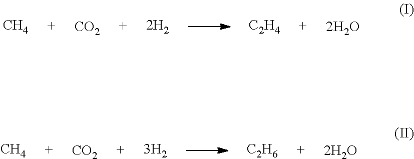

Compared to the conversion reaction without reactant gas CH₄ (e.g., 2 CO₂+6 H₂→C₂H₄+4 H₂O), the conversion reactions utilizing the reactant gas CH₄ may consume less H₂. Since the reactant gas in the disclosure contains CH₄, it may save the amount of H₂ and reduce the cost of synthesizing alkanes and olefins. Reaction Formulas (I) and (II) are merely an example. In practice, the major products may not only include C₂H₄ but also CₘH₂ₘ having a carbon number greater than 2 and CₙH₂ₙ₊₂ having a carbon number of 2 or more.

The inventors have found that employing a metal oxide catalyst having the general chemical formula of Fe₁MnₐM_{b}Oₓ to be particularly beneficial in synthesizing alkanes and olefins, i.e., arbitrarily replacing the elements in this general formula with other elements may reduce efficacy of the catalyst mixture. For example, if Mn, Ce, or La is replaced with another element such as Cu or Ni, then CH₄ cannot be converted to synthesize alkanes and olefins. If the amount of Mn is too low, the CO₂ and H₂ will react to form methane. If the amount of Mn is too high, the conversion rate will be decreased. If the amount of La or Ce is too low, the conversion rate will be decreased. If the amount of La or Ce is too high, the CO₂ and H₂ will react to form methane.

In embodiments, the method may include bringing a gaseous mixture of carbon dioxide, methane, and hydrogen into contact with the described catalyst mixture to form a product containing water and alkanes having a carbon number of 2 or more and olefins having a carbon number of 2 or more according to the reaction Formulas I and II above.

It should be understood that, despite the reaction Formulas I and II being the primary reactions, other hydrocarbons having a carbon number of more than 2, such as CₙH₂ₙ or CₙH₂ₙ₊₂, n≥3, may also be produced. Apart from that, other side reactions and byproducts are possible. As such, the products may contain byproducts and unreacted reactants. For example, the products may further include carbon monoxide and methane.

Without intending to be bound by theory, it is believed that the catalyst mixture functions under the disclosed conditions by facilitating the role of methane as a reactant and stabilizing the reaction. Conventional catalysts cannot be integrated into the disclosed reactions, as methane requires higher temperature (>700° C.) to react.

In some embodiments, the gaseous mixture has a gas hourly space velocity of 200 h⁻¹ to 3000 h⁻¹.

If the gas hourly space velocity of the gaseous mixture is too low, the unit catalyst efficiency may be relatively low and the economic efficiency may be insufficient. If the gas hourly space velocity of the gaseous mixture is too high, the contact time of the catalyst mixture and the gaseous mixture may be too short to form the alkanes and olefins.

In some embodiments, the molar ratio of the carbon dioxide and the methane in the gaseous mixture may be controlled to be within a specified range. For example, the molar ratio of the carbon dioxide and the methane in the gaseous mixture may be in a range of 1:0.2 to 1:4.

If the amount of the methane is too low, the conversion may consume more hydrogen. If the amount of the methane is too high, the non-reacted methane may be too much and the resource may be wasted.

In some embodiments, the molar ratio of the carbon dioxide and the hydrogen in the gaseous mixture may be controlled to be within a specified range. For example, the molar ratio of the carbon dioxide and the hydrogen in the gaseous mixture may be in a range of 1:0.5 to 1:4.

If the amount of the hydrogen is too low, the conversion rate of the carbon dioxide may be relatively low. If the amount of the hydrogen is too high, the non-reacted hydrogen may be too much and the resource will be wasted.

In some embodiments, the step of bringing the gaseous mixture into contact with the catalyst mixture is performed under a pressure of 10 bar to 40 bar at a temperature of 250° C. to 400° C.

If the temperature is too low, the carbon dioxide may not be converted efficiently. If the temperature is too high, it will easily form carbon monoxide rather than the alkanes and olefins.

If the pressure is too low, the alkanes and olefins having multiple carbon numbers may not be formed efficiently. If the pressure is too high, the energy consumption will be relatively high.

Below, exemplary embodiments will be described in detail so as to be easily realized by a person having ordinary knowledge in the art. The inventive concept may be embodied in various forms without being limited to the exemplary embodiments set forth herein. Descriptions of well-known parts are omitted for clarity.

### EXAMPLES

The following examples are provided for better understanding of exemplary embodiments. It should be appreciated that this disclosure is not intended to be limited to these specific examples and that one of ordinary skill in the art would understand that the following examples may be embodied in many different forms and should not be construed as limited to the exemplary embodiments set forth therein.

### Example 1

Iron nitrate, manganese nitrate, and lanthanum nitrate were weighed according to chemical stoichiometry and dissolved in water, and a pH value of this aqueous solution of the metal salts was adjusted by ammonia water to 9 and kept for 2 hours to produce a precipitate. The precipitate was collected by filtering, and the filtered cake was washed by water, baked, and then sintered (450° C.) to obtain a metal oxide catalyst Fe₁Mn_{0.43}La_{0.14}Oₓ (the element ratio was confirmed by ICP).

An aqueous solution of 18.5 mole of iron nitrate and ammonia water were stirred at high speed to mix for 3 minutes, and the pH value of the mixture was then adjusted to 9. The mixture was then continuously stirred for 120 minutes and then filtered out. The filtered cake was washed out with water and then baked to obtain an iron-based support such as porous FeO(OH)_{c}. The iron-based support was baked at 110° C. and then measured its weight (1730 g), thereby calculating the FeO(OH) content (48 mol %) and the FeO(OH)₂ content (52 mol %), i.e., c in the product FeO(OH)_{c} was 1.52. As shown by the Raman spectra, there are almost no signals of Fe₂O₃ and Fe₃O₄ in FeO(OH)_{c}. The obtained iron-based support such as FeO(OH)_{c} had a specific surface area of 249 m²/g (measured and calculated by BET method, such as nitrogen adsorption at constant temperature), a pore volume of 0.39 cm³/g (measured and calculated by BJH method, such as nitrogen adsorption at constant temperature), and an average pore size of 62 Å (measured and calculated by BJH method, such as nitrogen adsorption at constant temperature). The porous FeO(OH)_{c} was then immersed into a potassium carbonate aqueous solution, such that the potassium carbonate was loaded on the porous FeO(OH)_{c}. The above substance was then baked at 110° C. and shaped to obtain an iron-based catalyst, wherein the molar percentage of the FeO(OH)_{c} was 95 mol % (c=1.52), and that of the potassium carbonate was 5 mol %.

The metal oxide catalyst was sieved to obtain 60 mL of metal oxide catalyst powder with 8 mesh to 12 mesh. The iron-based catalyst was sieved to obtain 140 mL of iron-based catalyst powder with 8 mesh to 12 mesh. The metal oxide catalyst powder and the iron-based catalyst powder had a volume ratio of 3:7. The metal oxide catalyst powder and the iron-based catalyst powder were mixed and then filled into a 1-inch reactor. A gaseous mixture of carbon dioxide, methane, and hydrogen having a total gas hourly space velocity of 1200 hr⁻¹ (CO₂=1000 mL/min, CH₄=1000 mL/min, and H₂=2000 mL/min) was introduced into the reactor at a reaction temperature of 300° C. under a reaction pressure of 19.7 atm (20 bar). The gas composition of the product was analyzed by Gas Chromatography (such as simultaneously measured and calculated through a column Carboxen-1010 PLOT, 30 m*0.53 mm ID with TCD detector, and a column VB-1, 30 m*0.53 mm ID with FID detector). The same analysis would be applied in the following examples. As known from the analysis result and a calculation result of a conversion rate based on CO₂ (the same calculation would be applied in the following examples), the conversion rate of the carbon dioxide was 38.3%, the yield of C2+ alkanes and olefins was 30.3%, the selectivity of the C2+ alkanes and olefins was 79.1%, the yield of CH₄ was 4.1%, and the yield of carbon monoxide was 3.9%.

### Example 2

The metal oxide catalyst in Example 1 was sieved to obtain 60 mL of metal oxide catalyst powder with 8 mesh to 12 mesh. The iron-based catalyst in Example 1 was sieved to obtain 140 mL of iron-based catalyst powder with 8 mesh to 12 mesh. The metal oxide catalyst powder and the iron-based catalyst powder had a volume ratio of 3:7. The metal oxide catalyst powder and the iron-based catalyst powder were mixed and then filled into a 1-inch reactor. A gaseous mixture of carbon dioxide, methane, and hydrogen having a total gas hourly space velocity of 1800 hr⁻¹ (CO₂=1000 mL/min, CH₄=1000 mL/min, and H₂=4000 mL/min) was introduced into the reactor at a reaction temperature of 300° C. under a reaction pressure of 19.7 atm (20 bar). As known from the analysis result of gas composition of the product, the conversion rate of the carbon dioxide was 67.0%, the yield of C2+ alkanes and olefins was 53.7%, the selectivity of the C2+ alkanes and olefins was 80.1%, the yield of CH₄ was 5.8%, and the yield of carbon monoxide was 7.5%.

### Comparative Example 1 (the Reactant Gas was Free of CH₄)

The metal oxide catalyst in Example 1 was sieved to obtain 60 mL of metal oxide catalyst powder with 8 mesh to 12 mesh. The iron-based catalyst in Example 1 was sieved to obtain 140 mL of iron-based catalyst powder with 8 mesh to 12 mesh. The metal oxide catalyst powder and the iron-based catalyst powder had a volume ratio of 3:7. The metal oxide catalyst powder and the iron-based catalyst powder were mixed and then filled into a 1-inch reactor. A gaseous mixture of carbon dioxide and hydrogen having a total gas hourly space velocity of 900 hr⁻¹ (CO₂=1000 mL/min, CH₄=0 mL/min, and H₂=2000 mL/min) was introduced into the reactor at a reaction temperature of 300° C. under a reaction pressure of 19.7 atm (20 bar). As known from the analysis result of gas composition of the product, the conversion rate of the carbon dioxide was 22.8%, the yield of C2+ alkanes and olefins was 15.0%, the selectivity of the C2+ alkanes and olefins was 65.8%, the yield of CH₄ was 2.8%, and the yield of carbon monoxide was 5.1%. As shown above, the conversion rate of the carbon dioxide was relatively low when the reactant gas was free of methane.

### Comparative Example 2 (the Reactant Gas was Free of CH₄)

The metal oxide catalyst in Example 1 was sieved to obtain 60 mL of metal oxide catalyst powder with 8 mesh to 12 mesh. The iron-based catalyst in Example 1 was sieved to obtain 140 mL of iron-based catalyst powder with 8 mesh to 12 mesh. The metal oxide catalyst powder and the iron-based catalyst powder had a volume ratio of 3:7. The metal oxide catalyst powder and the iron-based catalyst powder were mixed and then filled into a 1-inch reactor. A gaseous mixture of carbon dioxide and hydrogen having a total gas hourly space velocity of 1500 hr⁻¹ (CO₂=1000 mL/min, CH₄=0 mL/min, and H₂=4000 mL/min) was introduced into the reactor at a reaction temperature of 300° C. under a reaction pressure of 19.7 atm (20 bar). As known from the analysis result of gas composition of the product, the conversion rate of the carbon dioxide was 40.6%, the yield of C2+ alkanes and olefins was 29.4%, the selectivity of the C2+ alkanes and olefins was 72.4%, the yield of CH₄ was 7.2%, and the yield of carbon monoxide was 4.0%. As shown above, the conversion rate of the carbon dioxide was relatively low and the yield of the C2+ alkanes and olefins was relatively low when the reactant gas was free of methane. In addition, more hydrogen was required when the reactant gas was free of methane, which consumed more hydrogen resource.

### Example 3

The metal oxide catalyst in Example 1 was sieved to obtain 100 mL of metal oxide catalyst powder with 8 mesh to 12 mesh. The iron-based catalyst in Example 1 was sieved to obtain 100 mL of iron-based catalyst powder with 8 mesh to 12 mesh. The metal oxide catalyst powder and the iron-based catalyst powder had a volume ratio of 5:5. The metal oxide catalyst powder and the iron-based catalyst powder were mixed and then filled into a 1-inch reactor.

### Example 3A

A gaseous mixture of carbon dioxide, methane, and hydrogen having a total gas hourly space velocity of 1575 hr⁻¹ (CO₂=1000 mL/min, CH₄=250 mL/min, and H₂=4000 mL/min) was introduced into the reactor at a reaction temperature of 280° C. under a reaction pressure of 19.7 atm (20 bar). As known from the analysis result of gas composition of the product, the conversion rate of the carbon dioxide was 19.44%, the yield of C2+ alkanes and olefins was 13.8%, the selectivity of the C2+ alkanes and olefins was 70.9%, the yield of CH₄ was 0.4%, and the yield of carbon monoxide was 5.3%.

### Example 3B

A gaseous mixture of carbon dioxide, methane, and hydrogen having a total gas hourly space velocity of 1650 hr⁻¹ (CO₂=1000 mL/min, CH₄=500 mL/min, and H₂=4000 mL/min) was introduced into the reactor at a reaction temperature of 280° C. under a reaction pressure of 19.7 atm (20 bar). As known from the analysis result of gas composition of the product, the conversion rate of the carbon dioxide was 16.63%, the yield of C2+ alkanes and olefins was 13.0%, the selectivity of the C2+ alkanes and olefins was 78.4%, the yield of CH₄ was −1.9%, and the yield of carbon monoxide was 5.6%.

### Example 3C

A gaseous mixture of carbon dioxide, methane, and hydrogen having a total gas hourly space velocity of 1800 hr⁻¹ (CO₂=1000 mL/min, CH₄=1000 mL/min, and H₂=4000 mL/min) was introduced into the reactor at a reaction temperature of 280° C. under a reaction pressure of 19.7 atm (20 bar). As known from the analysis result of gas composition of the product, the conversion rate of the carbon dioxide was 18.5%, the yield of C2+ alkanes and olefins was 16.5%, the selectivity of the C2+ alkanes and olefins was 89.4%, the yield of CH₄ was −4.6%, and the yield of carbon monoxide was 6.6%.

### Example 4

Iron nitrate, manganese nitrate, and cerium nitrate were weighed according to chemical stoichiometry and dissolved in water, and a pH value of this aqueous solution of the metal salts was adjusted by ammonia water to 9 and kept for 2 hours to produce a precipitate. The precipitate was collected by filtering, and the filtered cake was washed by water, baked, and then sintered (450° C.) to obtain a metal oxide catalyst Fe₁Mn_{0.43}Ce_{0.14}Oₓ (the element ratio was confirmed by ICP).

The metal oxide catalyst (Fe₁Mn_{0.43}Ce_{0.14}Oₓ) was sieved to obtain 100 mL of metal oxide catalyst powder with 8 mesh to 12 mesh. The iron-based catalyst in Example 1 (the molar percentage of the FeO(OH)_{c} was 95 mol % (c=1.52), and that of the potassium carbonate was 5 mol %) was sieved to obtain 100 mL of iron-based catalyst powder with 8 mesh to 12 mesh. The metal oxide catalyst powder and the iron-based catalyst powder had a volume ratio of 5:5. The metal oxide catalyst powder and the iron-based catalyst powder were mixed and then filled into a 1-inch reactor. A gaseous mixture of carbon dioxide, methane, and hydrogen having a total gas hourly space velocity of 1800 hr⁻¹ (CO₂=1000 mL/min, CH₄=1000 mL/min, and H₂=4000 mL/min) was introduced into the reactor at a reaction temperature of 280° C. under a reaction pressure of 19.7 atm (20 bar). As known from the analysis result of gas composition of the product, the conversion rate of the carbon dioxide was 19.7%, the yield of C2+ alkanes and olefins was 16.2%, the selectivity of the C2+ alkanes and olefins was 82.3%, the yield of CH₄ was −3.2%, and the yield of carbon monoxide was 6.7%.

### Comparative Example 3

The iron-based catalyst in Example 1 was sieved to obtain 200 mL of iron-based catalyst powder with 8 mesh to 12 mesh. The iron-based catalyst powder was filled into a 1-inch reactor. A gaseous mixture of carbon dioxide, methane, and hydrogen having a total gas hourly space velocity of 1800 hr⁻¹ (CO₂=1000 mL/min, CH₄=1000 mL/min, and H₂=4000 mL/min) was introduced into the reactor at a reaction temperature of 280° C. under a reaction pressure of 19.7 atm (20 bar). As known from the analysis result of gas composition of the product, the conversion rate of the carbon dioxide was 20.8%, the yield of C2+ alkanes and olefins was 10.3%, the selectivity of the C2+ alkanes and olefins was 49.6%, the yield of CH₄ was 3.9%, and the yield of carbon monoxide was 6.6%. As shown above, the yield of the C2+ alkanes and olefins could not be enhanced by utilizing iron-based catalyst alone with methane.

### Comparative Example 4

The metal oxide catalyst in Example 1 was sieved to obtain 200 mL of metal oxide catalyst powder with 8 mesh to 12 mesh. The metal oxide catalyst powder was filled into a 1-inch reactor. A gaseous mixture of carbon dioxide, methane, and hydrogen having a total gas hourly space velocity of 1800 hr⁻¹ (CO₂=1000 mL/min, CH₄=1000 mL/min, and H₂=4000 mL/min) was introduced into the reactor at a reaction temperature of 280° C. under a reaction pressure of 19.7 atm (20 bar). As known from the analysis result of gas composition of the product, the conversion rate of the carbon dioxide was 18.7%, the yield of C2+ alkanes and olefins was 0.0%, the selectivity of the C2+ alkanes and olefins was 0.0%, the yield of CH₄ was 0.0%, and the yield of carbon monoxide was 18.7%. As shown above, CH₄ could not react to form alkanes and olefins by utilizing the metal oxide catalyst alone.

### Comparative Example 5 (the Reactant Gas Without CH₄)

The metal oxide catalyst in Example 1 was sieved to obtain 100 mL of metal oxide catalyst powder with 8 mesh to 12 mesh. The iron-based catalyst in Example 1 was sieved to obtain 100 mL of iron-based catalyst powder with 8 mesh to 12 mesh. The metal oxide catalyst powder and the iron-based catalyst powder had a volume ratio of 5:5. The metal oxide catalyst powder and the iron-based catalyst powder were mixed and then filled into a 1-inch reactor. A gaseous mixture of carbon dioxide and hydrogen having a total gas hourly space velocity of 1500 hr⁻¹ (CO₂=1000 mL/min and H₂=4000 mL/min) was introduced into the reactor at a reaction temperature of 280° C. under a reaction pressure of 19.7 atm (20 bar). As known from the analysis result of gas composition of the product, the conversion rate of the carbon dioxide was 18.5%, the yield of C2+ alkanes and olefins was 12.8%, the selectivity of the C2+ alkanes and olefins was 70.2%, the yield of CH₄ was 3.1%, and the yield of carbon monoxide was 2.6%. As shown above, the yield of the C2+ alkanes and olefins could not be enhanced by the reactant gas free of methane.

### Comparative Example 6

Iron nitrate, copper nitrate, and lanthanum nitrate were weighed according to chemical stoichiometry and dissolved in water, and a pH value of this aqueous solution of the metal salts was adjusted by ammonia water to 9 and kept for 2 hours to produce a precipitate. The precipitate was collected by filtering, and the filtered cake was washed by water, baked, and then sintered (450° C.) to obtain a metal oxide catalyst Fe₁Cu_{0.43}La_{0.14}Oₓ (the element ratio was confirmed by ICP).

The metal oxide catalyst (Fe₁Cu_{0.43}La_{0.14}Oₓ) was sieved to obtain 100 mL of metal oxide catalyst powder with 8 mesh to 12 mesh. The iron-based catalyst in Example 1 was sieved to obtain 100 mL of iron-based catalyst powder with 8 mesh to 12 mesh. The metal oxide catalyst powder and the iron-based catalyst powder had a volume ratio of 5:5. The metal oxide catalyst powder and the iron-based catalyst powder were mixed and then filled into a 1-inch reactor. A gaseous mixture of carbon dioxide, methane, and hydrogen having a total gas hourly space velocity of 1800 hr⁻¹ (CO₂=1000 mL/min, CH₄=1000 mL/min, and H₂=4000 mL/min) was introduced into the reactor at a reaction temperature of 280° C. under a reaction pressure of 19.7 atm (20 bar). As known from the analysis result of gas composition of the product, the conversion rate of the carbon dioxide was 23.9%, the yield of C2+ alkanes and olefins was 13.7%, the selectivity of the C2+ alkanes and olefins was 57.4%, the yield of CH₄ was 3.9%, and the yield of carbon monoxide was 6.3%. As shown above, the effect of converting methane to synthesize C2+ alkanes and olefins was relatively poor and the selectivity of the C2+ alkanes and olefins was low when the catalyst mixture was a mixture of the metal oxide catalyst Fe₁Cu_{0.43}La_{0.14}Oₓ and the iron-based catalyst.

### Comparative Example 7

Iron nitrate, manganese nitrate, and copper nitrate were weighed according to chemical stoichiometry and dissolved in water, and a pH value of this aqueous solution of the metal salts was adjusted by ammonia water to 9 and kept for 2 hours to produce a precipitate. The precipitate was collected by filtering, and the filtered cake was washed by water, baked, and then sintered (450° C.) to obtain a metal oxide catalyst Fe₁Mn_{0.43}Cu_{0.14}Oₓ (the element ratio was confirmed by ICP).

The metal oxide catalyst (Fe₁Mn_{0.43}Cu_{0.14}Oₓ) was sieved to obtain 100 mL of metal oxide catalyst powder with 8 mesh to 12 mesh. The iron-based catalyst in Example 1 was sieved to obtain 100 mL of iron-based catalyst powder with 8 mesh to 12 mesh. The metal oxide catalyst powder and the iron-based catalyst powder had a volume ratio of 5:5. The metal oxide catalyst powder and the iron-based catalyst powder were mixed and then filled into a 1-inch reactor. A gaseous mixture of carbon dioxide, methane, and hydrogen having a total gas hourly space velocity of 1800 hr⁻¹ (CO₂=1000 mL/min, CH₄=1000 mL/min, and H₂=4000 mL/min) was introduced into the reactor at a reaction temperature of 280° C. under a reaction pressure of 19.7 atm (20 bar). As known from the analysis result of gas composition of the product, the conversion rate of the carbon dioxide was 23.5%, the yield of C2+ alkanes and olefins was 13.0%, the selectivity of the C2+ alkanes and olefins was 55.3%, the yield of CH₄ was 6.5%, and the yield of carbon monoxide was 4.0%. As shown above, the effect of converting methane to synthesize C2+ alkanes and olefins was relatively poor and the selectivity of the C2+ alkanes and olefins was low when the catalyst mixture was a mixture of the metal oxide catalyst Fe₁Mn_{0.43}Cu_{0.14}Oₓ and the iron-based catalyst.

It will be apparent to those skilled in the art that various modifications and variations can be made to the disclosed methods and materials. It is intended that the specification and examples be considered as exemplary only, with the true scope of the disclosure being indicated by the following claims and their equivalents.

What is claimed is:

1. A catalyst mixture comprising:
a mixture of a metal oxide catalyst and an iron-based catalyst,
wherein the metal oxide catalyst has a chemical formula of Fe₁MnₐM_{b}Oₓ, wherein M is Ce or La, a is 0.1 to 0.5, b is 0.1 to 0.3, and x is chemical stoichiometry,
wherein the iron-based catalyst comprises 70 mol % to 97 mol % of porous FeO(OH)_{c}, wherein 1<c<2, and 3 mol % to 30 mol % of an alkaline metal compound loaded onto the porous FeO(OH)_{c}.

2. The catalyst mixture as claimed in claim 1, wherein a is 0.1 to 0.43.

3. The catalyst mixture as claimed in claim 1, wherein b is 0.14 to 0.3.

4. The catalyst mixture as claimed in claim 1, wherein c satisfies 1<c≤1.52.

5. The catalyst mixture as claimed in claim 1, wherein the metal oxide catalyst and the iron-based catalyst have a volume ratio of 1:4 to 1:1.

6. The catalyst mixture as claimed in claim 1, wherein the porous FeO(OH)_{c} has a specific surface area of at least 100 m²/g.

7. The catalyst mixture as claimed in claim 1, wherein the porous FeO(OH)_{c} has a pore volume in a range of 0.2 cm³/g to 0.5 cm³/g.

8. The catalyst mixture as claimed in claim 1, wherein the porous FeO(OH)_{c} has an average pore size in a range of 40 Å to 70 Å.

9. A method of synthesizing alkanes and olefins, the method comprising:
bringing a gaseous mixture of carbon dioxide, methane, and hydrogen into contact with the catalyst mixture as claimed in claim 1 to form a product comprising water and at least one of alkanes having a carbon number of 2 or more and olefins having a carbon number of 2 or more.

10. The method as claimed in claim 9, wherein the product further comprises carbon monoxide and methane.

11. The method as claimed in claim 9, wherein the gaseous mixture has a gas hourly space velocity in a range of 200 h⁻¹ to 3000 h⁻¹.

12. The method as claimed in claim 9, wherein the carbon dioxide and the methane in the gaseous mixture have a molar ratio in a range of 1:0.2 to 1:4, and the carbon dioxide and the hydrogen in the gaseous mixture have a molar ratio in a range of 1:0.5 to 1:4.

13. The method as claimed in claim 9, wherein the step of bringing the gaseous mixture into contact with the catalyst mixture is performed under a pressure in a range of 10 bar to 40 bar, and at a temperature in a range of 250° C. to 400° .
